(19) United States
(12) Patent Application Publication
(10) Pub. No.: US 2022/0181017 A1
(43) Pub. Date: Jun. 9, 2022

(54) INFORMATION PROCESSING APPARATUS, NON-TRANSITORY COMPUTER READABLE MEDIUM, AND INFORMATION PROCESSING METHOD

(71) Applicant: TOYOTA JIDOSHA KABUSHIKI KAISHA, Aichi-ken (JP)

(72) Inventors: Toyokazu NAKASHIMA, Nagoya-shi (JP); Shunsuke SAGARA, Nisshin-shi (JP); Naoya OKA, Nagakute-shi (JP)

(21) Appl. No.: 17/457,913

(22) Filed: Dec. 7, 2021

(30) Foreign Application Priority Data
Dec. 8, 2020 (JP) 2020-203587

(51) Int. Cl. G16H 40/67; G16H 20/10; G16H 80/00; G08G 1/00; G06Q 50/30

(52) U.S. Cl. CPC G16H 40/67; G16H 20/10; G16H 80/00; G06Q 50/30; G08G 1/205; G08G 1/207; G08G 1/202

(57) ABSTRACT

An information processing apparatus includes a communication interface and a controller. The controller is configured to: based on positional information for a first user whose abnormal physical condition has been detected and positional information for a plurality of vehicles that a respective plurality of doctors are on board, determine to which one of the plurality of vehicles a signal that gives instructions to help the first user is to be transmitted; and based on the positional information for the first user, medical condition information for the first user, positional information for a plurality of medications, and efficacy information for the plurality of medications, determine which one of the plurality of medications is to be used for the first user.

### CROSS-REFERENCE TO RELATED APPLICATION

This application claims priority to Japanese Patent Application No.2020-203587, filed on Dec. 8, 2020, the entire contents of which are incorporated herein by reference.

### TECHNICAL FIELD

The present disclosure relates to an information processing apparatus, a program, and an information processing method.

### BACKGROUND

Apparatuses that assist in critical care of users with abnormal physical conditions are known. For example, Patent Literature 1 describes an emergency and critical care assistance system that includes a user terminal apparatus, a third party terminal apparatus, and an emergency and critical care assistance apparatus.

### CITATION LIST

### Patent Literature

PTL 1: JP 2012-222443 A

### SUMMARY

Know technology has room for improvement. For example, using medications to help users may reverse the abnormal physical conditions rapidly.

It would be helpful to provide an improved information processing apparatus, program, and information processing method.

An information processing apparatus according to an embodiment of the present disclosure includes a communication interface and a controller, wherein the controller is configured to: based on positional information for a first user whose abnormal physical condition has been detected and positional information for a plurality of vehicles that a respective plurality of doctors are on board, determine to which one of the plurality of vehicles a signal that gives instructions to help the first user is to be transmitted; and based on the positional information for the first user, medical condition information for the first user, positional information for a plurality of medications, and efficacy information for the plurality of medications, determine which one of the plurality of medications is to be used for the first user.

A program according to an embodiment of the present disclosure is configured to cause an information processing apparatus to execute operations including: based on positional information for a first user whose abnormal physical condition has been detected and positional information for a plurality of vehicles that a respective plurality of doctors are on board, determining to which one of the plurality of vehicles a signal that gives instructions to help the first user is to be transmitted; and based on the positional information for the first user, medical condition information for the first user, positional information for a plurality of medications, and efficacy information for the plurality of medications, determining which one of the plurality of medications is to be used for the first user.

An information processing method according to an embodiment of the present disclosure includes: based on positional information for a first user whose abnormal physical condition has been detected and positional information for a plurality of vehicles that a respective plurality of doctors are on board, determining to which one of the plurality of vehicles a signal that gives instructions to help the first user is to be transmitted; and based on the positional information for the first user, medical condition information for the first user, positional information for a plurality of medications, and efficacy information for the plurality of medications, determining which one of the plurality of medications is to be used for the first user.

According to the embodiments of the present disclosure, the improved information processing apparatus, program, and information processing method are provided.

### BRIEF DESCRIPTION OF THE DRAWINGS

In the accompanying drawings:

FIG. 1 illustrates a configuration of an information processing system according to an embodiment of the present disclosure;

FIG. 2 is a block diagram illustrating a detailed configuration of the information processing system of FIG. 1;

FIG. 3 illustrates an example of information registered on an information processing apparatus;

FIG. 4 illustrates an example of information registered on the information processing apparatus;

FIG. 5 illustrates an example of information registered on the information processing apparatus;

FIG. 6 is a sequence diagram illustrating an example of the operations of the information processing system of FIG. 1; and

FIG. 7 is a sequence diagram illustrating an example of the operations of the information processing system of FIG. 1.

### DETAILED DESCRIPTION

An embodiment of the present disclosure will be described below, with reference to the drawings. In the components illustrated in the drawings as described below, the same components are denoted by the same reference numerals.

### Configuration of Information Processing System

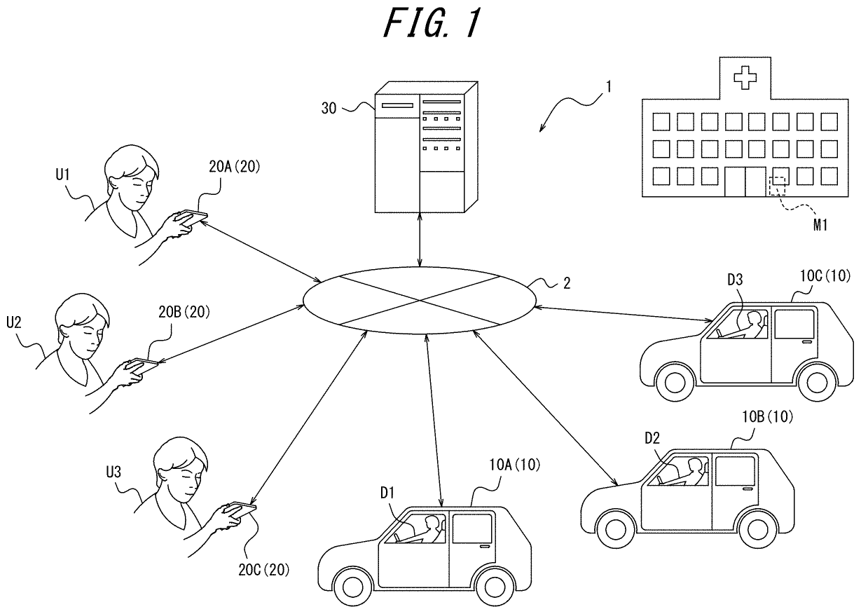

As illustrated in FIG. 1, an information processing system 1 according to an embodiment of the present disclosure includes a vehicle 10A, a vehicle 10B, a vehicle 10C, a terminal apparatus 20A, a terminal apparatus 20B, a terminal apparatus 20C, and an information processing apparatus 30.

In the following, the vehicle 10A, the vehicle 10B, and the vehicle 10C are also described collectively as “vehicles 10” unless particularly distinguished. FIG. 1 illustrates the information processing system 1 as including three vehicles 10. The information processing system 1, however, may include any number of vehicles 10.

In the following, the terminal apparatus 20A, the terminal apparatus 20B, and the terminal apparatus 20C are also described collectively as “terminal apparatuses 20” unless particularly distinguished. FIG. 1 illustrates the information processing system 1 as including three terminal apparatuses 20. The information processing system 1, however, may include any number of terminal apparatuses 20.

The vehicles 10, the terminal apparatuses 20, and the information processing apparatus 30 are communicable via a network 2. The network 2 may be any network, such as a mobile communication network or the Internet.

In each vehicle 10, a doctor is on board. Each vehicle 10 may be driven by a doctor. For example, the vehicle 10A is driven by a doctor D1. The vehicle 10B is driven by a doctor D2. The vehicle 10C is driven by a doctor D3. It is, however, sufficient for the doctors to be on board the vehicles 10, and they do not have to drive the vehicles 10.

Each vehicle 10 may be any type of automobile. The vehicle 10 is any type of automobile such as a gasoline vehicle, a diesel vehicle, a Hybrid Electric Vehicle (HEV), a Plug-in Hybrid Electric Vehicle (PHEV), a Battery Electric Vehicle (BEV), or a Fuel Cell Electric Vehicle (FCEV). The driving of each vehicle 10 may be automated at any level. The automation level is, for example, any one of Level 1 to Level 5 according to the level classification defined by the Society of Automotive Engineers (SAE). The vehicle 10 may be a dedicated Mobility as a Service (MaaS) vehicle.

Each terminal apparatus 20 is used by a user. For example, the terminal apparatus 20A is used by a user U1. The terminal apparatus 20B is used by a user U2. The terminal apparatus 20C is used by a user U3.

Each terminal apparatus 20 may have a dedicated application installed to provide a support service to support helping the user. The dedicated application that provides the support service is also referred to as a “support application” hereinafter.

The support service is a service that supports critical care of a user if an abnormal physical condition of the user is detected when they use the terminal apparatus 20. A user whose abnormal physical condition has been detected is also referred to as a “first user” hereinafter.

Each terminal apparatus 20 may be any apparatus, which can be connected to the network 2. The terminal apparatus 20 is, for example, a mobile phone, a smartphone, a tablet, a Personal computer (PC), or the like.

The information processing apparatus 30 provides the aforementioned support service. For example, as described later, the information processing apparatus 30 determines to which vehicle 10, among a plurality of vehicles 10, a signal that gives instructions to help the first user is to be transmitted, based on positional information or the like for the first user whose abnormal physical condition has been detected.

The information processing apparatus 30 may be a dedicated computer configured to function as a server, a general purpose personal computer, a cloud computing system, or the like.

### Configuration of Vehicle

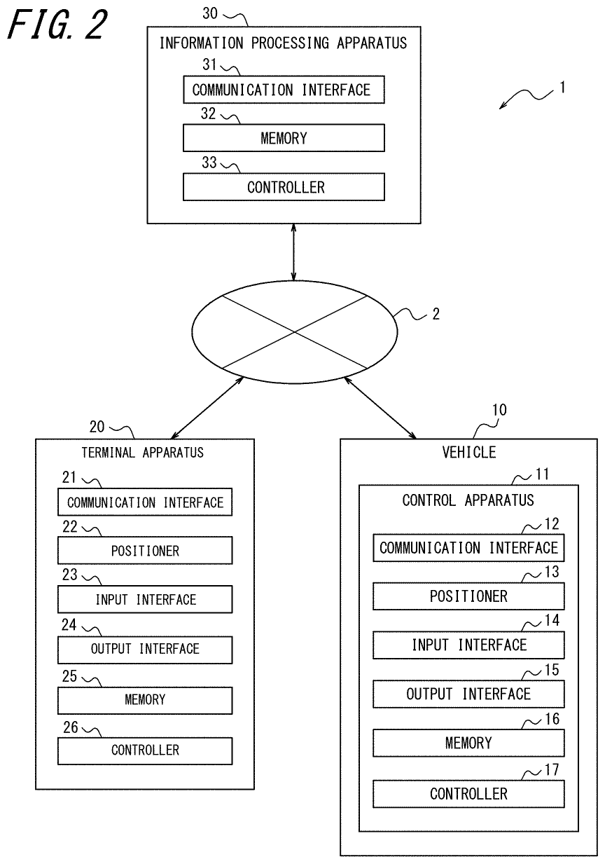

As illustrated in FIG. 2, each vehicle 10 includes a control apparatus 11. The control apparatus 11 may, for example, be a navigation device. The control apparatus 11 includes a communication interface 12, a positioner 13, an input interface 14, an output interface 15, a memory 16, and a controller 17.

The communication interface 12 is configured to include at least one communication module for connection to the network 2. For example, the communication module may include a communication module compliant with a mobile communication standard, such as the Long Term Evolution (LTE) standard, the 4th Generation (4G) standard, or the 5th Generation (5G) standard.

The positioner 13 is capable of acquiring positional information for the vehicle 10. The positioner 13 is configured to include at least one receiver module compliant with a satellite positioning system. The receiver module is, for example, a receiver module compliant with Global Positioning System (GPS). The receiver module is, however, not limited this example. The receiver module may be a receiver module compliant with any satellite positioning system.

The input interface 14 is capable of accepting an input from a user. The input interface 14 is configured to include at least one interface for input that is capable of accepting an input from a user. The interface for input may be a physical key, a capacitive key, a pointing device, a touch screen integrally provided with a display, a microphone, or the like.

The output interface 15 is capable of outputting data. The output interface 15 is configured to include at least one interface for output that is capable of outputting data. The interface for output may be a display, a speaker, or the like. The display may be a Liquid Crystal Display (LCD), an Electro-Luminescent (EL) display, or the like.

The memory 16 is configured to include at least one semiconductor memory, at least one magnetic memory, at least one optical memory, or a combination of at least two of these. The semiconductor memory is, for example, Random Access Memory (RAM), Read Only Memory (ROM), or the like. The RAM is, for example, Static Random Access Memory (SRAM), Dynamic Random Access Memory (DRAM), or the like. The ROM is, for example, Electrically Erasable Programmable Read Only Memory (EEPROM) or the like. The memory 16 may function as, for example, a main memory, an auxiliary memory, or a cache memory. The memory 16 stores data to be used for the operations of the vehicle 10 and data obtained by the operations of the vehicle 10.

The controller 17 is configured to include at least one processor, at least one dedicated circuit, or a combination thereof. The processor is a general purpose processor, such as a Central Processing Unit (CPU) or a Graphics Processing Unit (GPU), or a dedicated processor that is dedicated to specific processing. The dedicated circuit is, for example, a Field-Programmable Gate Array (FPGA) or an Application Specific Integrated Circuit (ASIC). The controller 17 executes processing related to the operations of the vehicle 10 while controlling the components of the vehicle 10. The controller 17 has a timekeeping function of obtaining the current time.

The functions of the vehicle 10 are realized by executing a control program according to the present embodiment by a processor corresponding to the controller 17. That is, the functions of the vehicle 10 are realized by software. The control program causes a computer to execute the operations of the vehicle 10, thereby causing the computer to function as the vehicle 10. That is, the computer functions as the vehicle 10 by executing the operations of the vehicle 10 according to the control program.

In the present disclosure, a “program” can be recorded on a computer readable non-transitory recording medium. The non-transitory computer readable recording medium is, for example, a magnetic recording device, an optical disc, a magneto-optical recording medium, or ROM. The program is distributed, for example, by selling, transferring, or renting a portable recording medium, such as a Digital Versatile Disc (DVD) or a Compact Disc Read Only Memory (CD-ROM), on which the program is recorded. The program may be stored in a storage of a server. The program may be distributed by being transferred from the server to another computer. The program may be provided as a program product.

In the present disclosure, a “computer” temporarily stores in the main memory, for example, a program recorded on a portable recording medium, or a program transferred from a server. Further, the computer reads the program stored in the main memory using a processor, and executes processes in accordance with the read program using the processor. The computer may read a program directly from the portable recording medium, and execute processes in accordance with the program. Each time a program is transferred from the server to the computer, the computer may execute processes in accordance with the received program in order. The computer may execute processes through a so-called Application Service Provider (ASP)-type service that realizes functions merely by execution of instructions and acquisition of results, without transferring the program from the server to the computer. Programs encompass information that is to be used for processing by an electronic computer and is thus equivalent to a program. For example, data that is not a direct command to a computer but has a property that regulates processing of the computer is “equivalent to a program” in this context.

Some or all of the functions of the vehicle 10 may be implemented by a dedicated circuit corresponding to the controller 17. That is, some or all of the functions of the vehicle 10 may be realized by hardware.

### Transmission Processing for Various Types of Information

A controller 17 executes transmission processing for transmitting, to the information processing apparatus 30, various types of information to be registered on the information processing apparatus 30. The various types of information to be registered on the information processing apparatus 30 include doctor identification information, doctor specialty information, medication identification information, and/or positional information for the vehicle 10. An example of the transmission processing for registration information is described below.

A controller 17 receives doctor identification information and doctor specialty information through the input interface 14. The doctor specialty information may be information that indicates what specialty the doctor holds. The doctor identification information may be information that can uniquely identify the doctor in the support service provided by the information processing apparatus 30. The doctor specialty information is, for example, information indicating that the doctor is a physician, a surgeon, an otolaryngologist, or the like. The doctor identification information and the doctor specialty information are inputted through the input interface 14 by a doctor who is on board the vehicle 10. The doctor inputs the above information through the input interface 14, for example, after boarding the vehicle 10 and before starting driving the vehicle 10. Upon receiving the doctor identification information and the doctor specialty information, the controller 17 transmits the doctor identification information and the doctor specialty information to the information processing apparatus 30 via the network 2 using the communication interface 12. The controller 17 stores, in the memory 16, the acquired doctor identification information.

The controller 17 receives medication identification information through the input interface 14. The medication identification information may be information that can uniquely identify the medication in the support service provided by the information processing apparatus 30. The medication identification information may be, for example, information that indicates an identification code of the medication that is used in a medical institution or the like, or a product name of the medication. The medication identification information is inputted through the input interface 14 by the doctor who is on board the vehicle 10. The doctor inputs the medication identification information through the input interface 14, for example, after boarding the vehicle 10 and before starting driving the vehicle 10. The doctor inputs identification information for a medication in their possession through the input interface 14 for the support service. The doctor is in possession of the medication for any purpose. For example, the doctor is in possession of the medication so as to provide the medication to a patient or to prepare for an acute seizure caused by a medical condition of the doctor themselves. Upon accepting the medication identification information, the controller 17 transmits the medication identification information, together with the doctor identification information, to the information processing apparatus 30 via the network 2 using the communication interface 12. The controller 17 may accept the doctor identification information, which is transmitted together with the medication identification information, through the input interface 14, or may retrieve it from the memory 16.

Here, after transmission processing for registration information is executed by the control apparatus 11, the doctor starts driving the vehicle 10. In a case in which the doctor always boards the same vehicle 10, doctor identification information and doctor specialty information may be stored in advance in the memory 16. In this case, the controller 17 retrieves doctor identification information and doctor specialty information from the memory 16 when operation of the vehicle 10 is started, and transmits the retrieved doctor identification information and doctor specialty information to the information processing apparatus 30 via the network 2 using the communication interface 12. Further, in a case in which a medication is always kept in the vehicle 10, medication identification information may be stored in advance in the memory 16. In this case, the controller 17 retrieves medication identification information from the memory 16 when operation of the vehicle 10 is started. The controller 17 transmits the medication identification information, together with the doctor identification information, to the information processing apparatus 30 via the network 2 using the communication interface 12. The controller 17 may accept the doctor identification information, which is transmitted together with the medication identification information, through the input interface 14, or may retrieve it from the memory 16.

While the vehicle 10 is travelling, the controller 17 acquires positional information for the vehicle 10 using the positioner 13. Upon acquiring the positional information for the vehicle 10, the controller 17 retrieves doctor identification information from the memory 16. The controller 17 transmits the positional information for the vehicle 10, together with the doctor identification information, to the information processing apparatus 30 via the network 2 using the communication interface 12.

The controller 17 may acquire positional information for the vehicle 10 and transmit the acquired positional information for the vehicle 10, together with the doctor identification information, to the information processing apparatus 30, at any time interval. The time interval may be set in advance based on the average speed of the vehicle 10, or may be changed appropriately in accordance with the speed of the vehicle 10. In a case in which the time interval is changed appropriately, the controller 17 may make the time interval shorter when the speed of the vehicle 10 is high than when the speed of the vehicle 10 is low.

The controller 17 may acquire a time at which positional information for the vehicle 10 is acquired as a travel time, by the timekeeping function of the controller 17. The controller 17 may transmit the positional information for the vehicle 10 and the travel time, together with the doctor identification information, to the information processing apparatus 30 via the network 2 using the communication interface 12. The controller 17 may acquire positional information for the vehicle 10 and a travel time and transmit the positional information for the vehicle 10 and the travel time, together with the doctor identification information, to the information processing apparatus 30, at any time interval as described above.

### Support Processing

While a vehicle 10 is travelling, the controller 17 executes support processing to support helping the first user based on a signal received from the information processing apparatus 30. As described above, the first user refers to a user whose abnormal physical condition has been detected. An example of the support processing is described below.

A controller 17 receives a signal that gives instructions to help the first user, from the information processing apparatus 30 via the network 2 using the communication interface 12. The signal that gives instructions to help the first user may include identification information for the first user. Upon receiving the signal, the controller 17 causes the output interface 15 to output information that gives instructions to help the first user. The information that gives the instructions to help the first user includes the identification information for the first user. Upon receiving positional information for the first user, together with the signal that gives instructions to help the first user, the controller 17 may cause the output interface 15 to output the positional information for the first user, together with information that gives instructions to help the first user. By the information being outputted from the output interface 15, a doctor driving the vehicle 10 can be aware that the first user with an abnormal physical condition is in the vicinity of the vehicle 10. The doctor can drive the vehicle 10 to the first user.

### Configuration of Terminal Apparatus

As illustrated in FIG. 2, a terminal apparatus 20 includes a communication interface 21, a positioner 22, an input interface 23, an output interface 24, a memory 25, and a controller 26. The terminal apparatus 20 may further include any sensor, such as an accelerometer or a biometric sensor.

As is the case with the communication interface 12, the communication interface 21 is configured to include at least one communication module for connection to the network 2.

The positioner 22 is capable of acquiring positional information for the terminal apparatus 20. As is the case with the positioner 13, the positioner 22 is configured to include at least one receiver module compliant with a satellite positioning system.

The input interface 23 is capable of accepting an input from a user. As is the case with the input interface 14, the input interface 23 is configured to include at least one interface for input that is capable of accepting an input from the user.

The output interface 24 is capable of outputting data. As is the case with the output interface 15, the output interface 24 is configured to include at least one interface for output that is capable of outputting data.

As is the case with the memory 16, the memory 25 is configured to include one magnetic memory, at least one optical memory, or a combination of at least two of these. The memory 25 may function as, for example, a main memory, an auxiliary memory, or a cache memory. The memory 25 stores data to be used for the operations of the terminal apparatus 20 and data obtained by the operations of the terminal apparatus 20. The memory 25 stores, for example, identification information for a user of the terminal apparatus 20. The user identification information may be information that can uniquely identify the user in the support service provided by the information processing apparatus 30.

As is the case with the controller 17, the controller 26 is configured to include at least one processor, at least one dedicated circuit, or a combination thereof. The controller 26 executes processes related to the operations of the terminal apparatus 20 while controlling the components of the terminal apparatus 20. The controller 26 has a timekeeping function of obtaining the current time.

The functions of the terminal apparatus 20 are realized by executing a terminal program, including a program for the support application according to the present embodiment, by a processor corresponding to the controller 26. That is, the functions of the terminal apparatus 20 are realized by software. The terminal program causes a computer to execute the operations of the terminal apparatus 20, thereby causing the computer to function as the terminal apparatus 20. That is, the computer executes the operations of the terminal apparatus 20 in accordance with the terminal program to thereby function as the terminal apparatus 20.

Some or all of the functions of the terminal apparatus 20 may be implemented by a dedicated circuit corresponding to the controller 26. That is, some or all of the functions of the terminal apparatus 20 may be realized by hardware.

### Transmission Processing for Various Types of Information

A controller 26 executes transmission processing for transmitting, to the information processing apparatus 30, various types information to be registered on the information processing apparatus 30. The various types of information to be registered on the information processing apparatus 30 include user identification information, user medical condition information, medication identification information, and/or user positional information. An example of the transmission processing for registration information is described below.

A controller 26 receives user medical condition information through the input interface 23. The user medical condition information is inputted by the user through the input interface 23. The user inputs their medical condition information through the input interface 23. The user medical condition information is, for example, information indicating the name of a disease that the user suffers from. Upon accepting an input indicating the user medical condition information, the controller 26 retrieves user identification information from the memory 25. The controller 26 transmits the user medical condition information, together with the user identification information, to the information processing apparatus 30 via the network 2 using the communication interface 21.

The controller 26 accepts medication identification information through the input interface 23. The medication identification information is inputted by the user through the input interface 23. The user inputs identification information for a medication in their possession through the input interface 23 for the support service. The user is in possession of the medication for any purpose. For example, the user is in possession of the medication to prepare for an acute seizure caused by a medical condition of the user themselves, to sell the medication, or to provide treatment using the medication. Upon accepting the medication identification information, the controller 26 retrieves user identification information from the memory 25. The controller 26 transmits the medication identification information, together with the user identification information, to the information processing apparatus 30 via the network 2 using the communication interface 21.

The controller 26 acquires positional information for the terminal apparatus 20 using the positioner 22, for example, while power supply to the terminal apparatus 20 is in an on state. The controller 26 considers the positional information for the terminal apparatus 20 to be user positional information. Upon acquiring the user positional information, the controller 26 retrieves user identification information from the memory 25. The controller 26 transmits the user positional information, together with the user identification information, to the information processing apparatus 30 via the network 2 using the communication interface 21.

The controller 26 may acquire user positional information and transmit the user positional information, together with the user identification information, to the information processing apparatus 30 at any time interval. The time interval may be set in advance based on an average value of the walking speed of the user, or may be changed appropriately in accordance with the walking speed of the user. In a case in which the time interval is changed appropriately, the controller 26 may make the time interval shorter when the walking speed of the user is faster than when the walking speed of the user is slower. The controller 26 may acquire the walking speed of the user based on a detection value detected by any sensor, such as an accelerometer, included in the terminal apparatus 20.

The controller 26 may acquire a time at which user positional information is acquired as a travel time, by the timekeeping function of the controller 26. The controller 26 may transmit the user positional information and the travel time, together with user identification information, to the information processing apparatus 30 via the network 2 using the communication interface 21. The controller 26 may acquire user positional information and a travel time and transmit the user positional information and the travel time, together with the user identification information, to the information processing apparatus 30, at any time interval as described above.

### Report Processing

A controller 26 executes, upon detecting an abnormal physical condition of a user, report processing for transmitting a signal indicating the abnormal physical condition to the information processing apparatus 30. An example of the reporting processing is described below.

A controller 26 receives an input indicating an abnormal physical condition of a user through the input interface 23. By receiving the input indicating the abnormal physical condition of the user, the controller 26 detects the abnormal physical condition of the user. The input is made through the input interface 23 by the user whose physical condition has deteriorated due to a seizure caused by the medical condition. In a case in which the input interface 23 is a touchscreen provided integrally with a display, a call button provided by the support application may be shown on the display. In this case, the user whose physical condition has deteriorated presses the call button. The controller 26 accepts an operation of pressing the call button as an input indicating an abnormal physical condition of the user, through the input interface 23.

The controller 26 may detect an abnormal physical condition of the user based on a detection value detected by a biometric sensor included in the terminal apparatus 20. The detection value corresponds, for example, to body temperature, pulse rate, heart rate, respiration rate, or the like. The controller 26 detects an abnormal physical condition of the user if the biometric sensor of the terminal apparatus 20 detects a value that is outside a predetermined setting range. The setting range may be set appropriately based on a normal detection value of the user.

Upon detecting an abnormal physical condition of the user, the controller 26 acquires positional information for the terminal apparatus 20 using the positioner 22 and retrieves user identification information from the memory 25. The controller 26 transmits a signal indicating the abnormal physical condition and the positional information for the terminal apparatus 20, together with the user identification information, to the information processing apparatus 30 via the network 2 using the communication interface 21.

### Configuration of Information Processing Apparatus

As illustrated in FIG. 2, the information processing apparatus 30 includes a communication interface 31, a memory 32, and a controller 33.

The communication interface 31 is configured to include at least one communication module for connection to the network 2. For example, the communication module is a communication module compliant with a standard such as a wired Local Area Network (LAN) or a wireless LAN. The communication interface 31 is connectable to the network 2 via the wired LAN or the wireless LAN using the communication module.

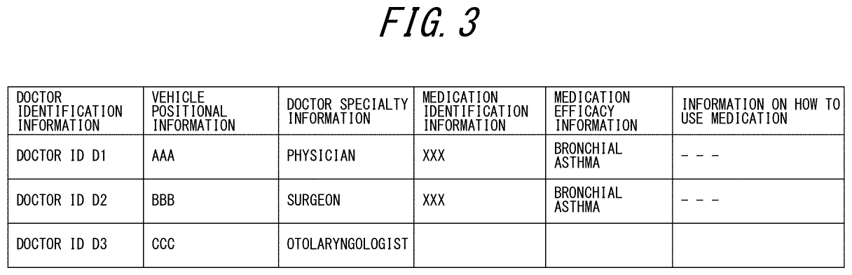

As is the case with the configuration of the memory 16, the memory 32 is configured to include at least one semiconductor memory, at least one magnetic memory, at least one optical memory, or a combination of at least two of these. The memory 32 may function as a main memory, an auxiliary memory, a cache memory, or the like. The memory 32 stores data to be used for the operations of the information processing apparatus 30 and data obtained by the operations of the information processing apparatus 30. The memory 32 stores information as illustrated in later-described FIG. 3 to FIG. 5. In the memory 32, for example, information on connection destinations of terminal apparatuses 20 used by users is stored in association with identification information for the users.

As is the case with the configuration of the controller 17, the controller 33 is configured to include at least one processor, at least one dedicated circuit, or a combination thereof. The controller 33 executes processes related to the operations of the information processing apparatus 30 while controlling the components of the information processing apparatus 30. The controller 33 may have a timekeeping function of obtaining the current time.

The functions of the information processing apparatus 30 may be implemented by executing a processing program according to the present embodiment by a processor corresponding to the controller 33. That is, the functions of the information processing apparatus 30 may be implemented by software. The processing program enables a computer to function as the information processing apparatus 30 by causing the computer to execute the operations of the information processing apparatus 30. That is, the computer functions as the information processing apparatus 30, by executing the operations of the information processing apparatus 30 in accordance with the processing program.

Some or all of the functions of the information processing apparatus 30 may be implemented by a dedicated circuit corresponding to the controller 33. That is, some or all of the functions of the information processing apparatus 30 may be implemented by hardware.

### Registration Processing for Various Types of Information

The controller 33 receives various types of information from a vehicle 10 or a terminal apparatus 20 via the network 2 using the communication interface 31. The controller 33 executes registration processing for registering various types of information by storing the received various types of information in the memory 32. An example of the registration processing is described below.

The controller 33 receives doctor identification information and doctor specialty information from a vehicle 10 via the network 2 using the communication interface 31. The controller 33 stores, in the memory 32, the doctor specialty information in association with the doctor identification information, thereby registering the doctor specialty information in association with the doctor identification information.

The controller 33 receives medication identification information, together with doctor identification information, from a vehicle 10 via the network 2 using the communication interface 31. The controller 33 stores, in the memory 32, the medication identification information in association with the doctor identification information, thereby registering the medication identification information in association with the doctor identification information.

The controller 33 receives positional information for a vehicle 10, together with doctor identification information, from the vehicle 10 via the network 2 using the communication interface 31. The controller 33 stores, in the memory 32, the positional information for the vehicle 10 in association with the doctor identification information, thereby registering the positional information for the vehicle 10 in association with the doctor identification information. In a case in which the controller 33 receives positional information for a vehicle 10 and a travel time, together with doctor identification information, the controller 33 may register positional information for the vehicle 10 received together with the latest travel time.

For example, the doctor ID “D1” as illustrated in FIG. 3 is identification information for the doctor D1. The doctor ID “D1” is associated with the positional information “AAA” for the vehicle 10A as the “vehicle positional information.” The doctor ID “D1” is also associated with the specialty information “physician” for the doctor D1 as the “doctor specialty information.” The doctor ID “D1” is also associated with the medication identification information “XXX” as the “medication identification information.”

For example, the doctor ID “D2” as illustrated in FIG. 3 is identification information for the doctor D2. The doctor ID “D2” is associated with the positional information “BBB” for the vehicle 10B as the “vehicle positional information.” The doctor ID “D2” is also associated with the specialty information “surgeon” for the doctor D2 as the “doctor specialty information.” The doctor ID “D2” is also associated with the medication identification information “XXX” as the “medication identification information.”

For example, the doctor ID “D3” as illustrated in FIG. 3 is identification information for the doctor D3. The doctor ID “D3” is associated with the positional information “CCC” for the vehicle 10C as the “vehicle positional information.” The doctor ID “D3” is also associated with the specialty information “otolaryngologist” for the doctor D3 as the “doctor specialty information.”

The controller 33 receives user medical condition information, together with user identification information, from a terminal apparatus 20 via the network 2 using the communication interface 31. The controller 33 stores, in the memory 32, the user medical condition information in association with the user identification information, thereby registering the user medical condition information in association with the user identification information.

The controller 33 receives medication identification information, together with user identification information, from a terminal apparatus 20 via the network 2 using the communication interface 31. The controller 33 stores, in the memory 32, the medication identification information in association with the user identification information, thereby registering the medication identification information in association with the user identification information.

The controller 33 receives user positional information, together with user identification information, from a terminal apparatus 20 via the network 2 using the communication interface 31. The controller 33 stores, in the memory 32, the user positional information in association with the user identification information, thereby registering the user positional information in association with the user identification information. In a case in which the controller 33 receives positional information for a terminal apparatus 20 and a travel time, together with user identification information, the controller 33 may register user positional information received together with the latest travel time.

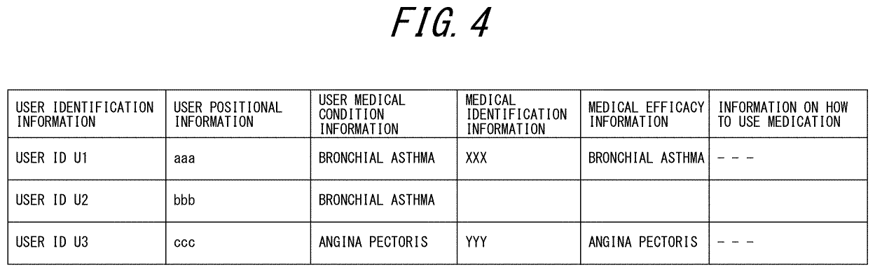

For example, the user ID “U1” as illustrated in FIG. 4 is identification information for the user U1. The user ID “U1” is associated with the positional information “aaa” for the user U1 as the “user positional information.” The user ID “U1” is also associated with the medical condition information “bronchial asthma” for the user U1 as the “user medical condition information.” The user ID “U1” is also associated with the medication identification information “XXX” as the “medical identification information.”

For example, the user ID “U2” as illustrated in FIG. 4 is identification information for the user U2. The user ID “U2” is associated with the positional information “bbb” for the user U2 as the “user positional information.” The user ID “U2” is also associated with the medical condition information “bronchial asthma” for the user U2 as the “user medical condition information.”

For example, the user ID “U3” as illustrated in FIG. 4 is identification information for the user U3. The user ID “U3” is associated with the positional information “ccc” for the user U3 as the “user positional information.” The user ID “U3” is also associated with the medical condition information “angina pectoris” for the user U3 as the “user medical condition information.” The user ID “U3” is also associated with the medication identification information “YYY” as the “medical identification information.”

Upon registration of medication identification information, the controller 33 acquires, for identification information for each medication registered, medication efficacy information and information on how to use the medication. For example, the controller 33 transmits identification information for respective medications to an external server or the like via the network 2 using the communication interface 31. After transmitting the identification information for the respective medications, the controller 33 acquires efficacy information for the medications and information on how to use the medications by receiving from the external server or the like via the network 2 using the communication interface 31. The controller 33 stores, in the memory 32, the acquired medication efficacy information and information on how to use the medications in association with the medication identification information, thereby registering the medication efficacy information and the information on how to use the medications in association with the medication identification information. Medication efficacy information may be information indicating one or more names of diseases for which the medication is effective. For example, in a case in which a medication is effective for bronchial asthma, its medication efficacy information is information indicating bronchial asthma. In a case in which a single medication is effective for two or more diseases, the medication efficacy information includes two or more pieces of information indicating the names of diseases for which the medication is effective. Information on how to use a medication includes, for example, information on the dosage of the medication and information on ways to administer the medication. Examples of ways to administer the medication to a user include swallowing the medication, applying the medication to the skin, or injecting the medication.

For example, the medication identification information “XXX” as illustrated in FIG. 3 and FIG. 4 is associated with the medication efficacy information “bronchial asthma” and with information on how to use the medication. The mediation identification information “YYY” is associated with the medication efficacy indication “angina pectoris” and with information on how to use the medication.

The controller 33 may receive medical device identification information, medical device positional information, information on the type of the medical device, information on the date and time when the medical device can be used, and information on how to use the medical device from an external server or the like via the network 2 using the communication interface 31. The medical device may be a piece of equipment used to diagnose, treat, or prevent a human disease. The controller 33 may store, in the memory 32, the medical device positional information, the information on the type of the medical device, the information on the date and time when the medical device can be used, and the information on how to use the medical device, in association with the medical device identification information. The controller 33 registers the above information in association with the medical device identification information, by storing, in the memory 32, the above information in association with the medical device identification information.

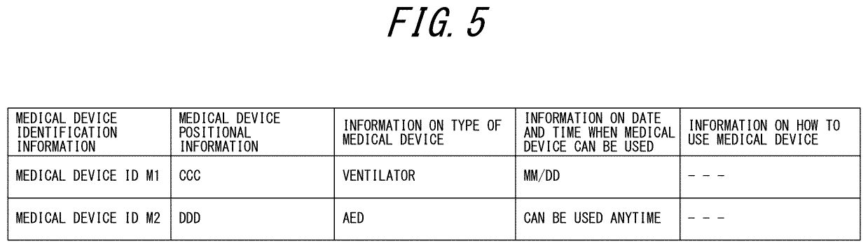

For example, the device ID “M1” as illustrated in FIG. 5 is identification information for a device M1 as illustrated in FIG. 1. The device M1 is a ventilator. The device M1 is installed at a hospital. The device ID “M1” is associated with the positional information “CCC” for the device M1 as the “medical device positional information.” The positional information “CCC” is, for example, positional information for the hospital at which the device M1 is installed. The device ID “M1” is also associated with information indicating the type of the device M1 “ventilator” as the “information on type of medical device.” The device ID “M1” is also associated with information indicating the date “MM/DD” as the “information on date and time when medical device can be used.” The date “MM/DD” is a day on which the hospital with the device M1 is closed. The device ID “M1” is also associated with information on how to use the device M1.

For example, the device ID “M2” as illustrated in FIG. 5 is identification information for an Automated External Defibrillator (AED) M2. The device ID “M2” is associated with the positional information “DDD” for the device M2 as the “medical device positional information.” The device ID “M2” is associated with the information indicating “can be used anytime” as the “information on date and time when medical device can be used.” This means that the medical device M2 can be used anytime. The device ID “M2” is also associated with information on how to use the device M2.

### Detection Processing

The controller 33 executes detection processing for detecting an abnormal physical condition of a user. For example, the controller 33 receives a signal indicating an abnormal physical condition of a user from the terminal apparatus 20 via the network 2 using the communication interface 31. By receiving a signal indicating an abnormal physical condition of a user from the terminal apparatus 20, the controller 33 detects the abnormal physical condition of the user. For example, by receiving a signal indicating an abnormal physical condition of a user from the terminal apparatus 20B, the controller 33 detects the abnormal physical condition of the user U2. As described earlier, a user whose abnormal physical condition has been detected is also referred to as the “first user.”

### Determination Processing

Upon detection of an abnormal physical condition of the first user, the controller 33 executes determination processing for determining to which vehicle 10, among a plurality of vehicles 10, a signal that gives instructions to help the first user is to be transmitted and for determining which one of a plurality of medications is to be used for the first user. The signal that gives instructions to help the first user may include identification information for the first user.

The controller 33 determines to which vehicle 10 transmission is to be performed, based on positional information for the first user and positional information for the plurality of vehicles 10 that a respective plurality of doctors are on board. Further, the controller 33 determines which one of the plurality of medications is to be used for the first user, based on the positional information for the first user, medical condition information for the first user, positional information for the plurality of medications, and efficacy information for the plurality of medications.

Here, as the positional information for the plurality of vehicles 10 that the respective plurality of doctors are on board, the controller 33 may use positional information for the plurality of vehicles 10 registered in association with doctor identification information.

Further, as positional information for at least one medication included in the positional information for the plurality of medications, the controller 33 may use positional information for a second user who is presumed to be in possession of the at least one medication. For example, in a case in which medication identification information is registered in association with user identification information, the controller 33 infers that the user is in possession of the medication and uses positional information for the user as positional information for the medication. In other words, in a case in which medication identification information is registered in association with user identification information, the controller 33 identifies the user as the second user who is presumed to be in possession of the medication. For example, in FIG. 4, the controller 33 uses the positional information “aaa” for the user U1 as positional information for a medication with the identification information “XXX.” Further, the controller 33 uses the positional information “ccc” for the user U3 as positional information for a medication with the identification information “YYY.”

Additionally, the controller 33 may identify the second user who is presumed to be in possession of a medication based on user medical condition information. For example, it is assumed that the user medical condition information is information indicating the name of a disease. In this case, information indicating the names of diseases and identification information for medications used to treat the diseases with these names may be stored in the memory 32 in association with each other. In a case in which information indicating the name of a disease corresponding to user medical condition information is stored in association with medication identification information in the memory 32, the controller 33 infers that the user is in possession of the medication and identifies the user as the second user who is presumed to be in possession of the medication. Upon identifying the user as the second user who is presumed to be in possession of the medication, the controller 33 uses positional information for the second user as positional information for the medication. For example, it is assumed that information indicating the name of the disease “bronchial asthma” and the medication identification information “XXX” are stored in the memory 32 in association with each other. In this case, the controller 33 identifies the user U2 as the second user who is presumed to be in possession of the medication with the identification information “XXX”, based on the medical condition information “bronchial asthma” for the user U2. The controller 33 uses the positional information “bbb” for the user U2 as positional information for a medication with the identification information “XXX.”

Further, as positional information for at least one medication included in the positional information for the plurality of medications, the controller 33 may use positional information for a vehicle 10 that a doctor who is presumed to be in possession of the at least one medication is on board. In other words, the controller 33 may use positional information for a vehicle 10 registered in association with identification information for a doctor who is presumed to be in possession of the at least one medication. In a case in which medication identification information is registered in association with doctor identification information, the controller 33 infers that the doctor is in possession of the medication. For example, in the configuration illustrated in FIG. 3, the controller 33 infers that the doctor D1 is in possession of a medication with the identification information “XXX.” The controller 33 also infers that the doctor D2 is in possession of the medication with the identification information “XXX.” The controller 33 uses the positional information “AAA” for the vehicle 10A and the positional information “BBB” for the vehicle 10B as positional information for the medication with the identification information “XXX.”

An example of the determination processing is described below.

### EXAMPLE 1: MEDICATION DETERMINATION PROCESSING

The controller 33 determines a medication in a plurality of medications that is selected based on medical condition information for the first user and efficacy information for the plurality of medications and that is located closest to the position of the first user, as a medication to be used for the first user. Here, an abnormal physical condition of the first user is often caused by the medical condition of the user. By selecting a medication based on the medical condition information for the first user and on the efficacy information for the plurality of medications, the abnormal physical condition of the first user can be reversed rapidly using the medication. For example, it is assumed that the medical condition information for the first user is information indicating the name of a disease and the efficacy information for the medications is information indicating the names of diseases for which the medications are effective. In this case, the controller 33 selects, from the plurality of medications, a medication that is associated with efficacy information indicating the name of a disease that is the same as that indicated by the medical condition information for the first user. Then, the controller 33 determines, with respect to the selected medication, a medication that is located closest to the position of the first user as the medication to be used for the first user, based on positional information for the first user and positional information for the medications.

For example, it is assumed that the first user is the user U2. In the configuration illustrated in FIG. 4, medical condition information for the user U2 is information indicating the name of the disease “bronchial asthma.” Further, efficacy information for a medication with the identification information “XXX” indicates the name of the disease “bronchial asthma.” The controller 33 selects the medication with the identification information “XXX” from the plurality of medications. Here, in the configuration illustrated in FIG. 3 and FIG. 4, positional information for the medication with the identification information “XXX” includes the positional information “AAA” for the vehicle 10A, the positional information “BBB” for the vehicle 10B, and the positional information “aaa” for the user U1. In the configuration illustrated in FIG. 3 and FIG. 4, it is assumed that the position of the user U1, among those of the vehicle 10A, the vehicle 10B and the user U1, is closest to the position of the user U2, that is, the first user. The controller 33 determines that the medication with the identification information “XXX” in possession of the user U1 is to be used for the user U2.

In a case in which positional information for the determined medication to be used for the first user is positional information for the above second user, the controller 33 transmits, to a terminal apparatus 20 used by the second user, a signal that gives instructions to provide the medication and positional information for the first user, via the network 2 using the communication interface 31. The signal that gives instructions to provide the medication may include identification information for the medication instructed to be provided. For example, it is assumed that the medication having the identification information “XXX” in possession of the user U1 as illustrated in FIG. 4 has been determined to be used for the user U2 as the first user. Positional information for this medication is the positional information for the user U1. The controller 33 transmits, to the terminal apparatus 20A used by the user U1, a signal that gives instructions to provide the medication and the positional information for the first user, via the network 2 using the communication interface 31. With the above configuration, the controller 26 of the terminal apparatus 20 can receive the signal that gives instructions to provide the medication and the positional information for the first user, from the information processing apparatus 30 via the network 2 using the communication interface 21. The controller 26 can cause the output interface 24 to output information that gives instructions to provide the medication and the positional information for the first user. The information that gives instructions to provide the medication may include identification information for the medication instructed to be provided. As a result of the information that gives instructions to provide the medication and the positional information for the first user being outputted from the output interface 24, the user can travel to the first user with the medication so as to reverse an abnormal physical condition of the first user.

In a case in which positional information for the determined medication to be used for the first user is positional information for a vehicle 10, the controller 33 transmits a signal that gives instructions to provide the medication and positional information for the first user to the vehicle 10 using the communication interface 31. With the above configuration, the controller 17 of the vehicle 10 can receive the signal that gives instructions to provide the medication and the positional information for the first user, from the information processing apparatus 30 via the network 2 using the communication interface 12. The controller 17 can cause the output interface 15 to output information that gives instructions to provide the medication and the positional information for the first user. The information that gives instructions to provide the medication may include identification information for the medication instructed to be provided. As a result of the information that gives instructions to provide the medication and the positional information for the first user being outputted from the output interface 15, a doctor driving the vehicle 10 can travel to the first user with the medication so as to reverse the abnormal physical condition of the first user.

### EXAMPLE 2: VEHICLE DETERMINATION PROCESSING

The controller 33 determines a vehicle 10 in a plurality of vehicles 10 that is located within a predetermined setting range from the position of the first user, as a vehicle 10 to which a signal that gives instructions to help the first user is to be transmitted. The setting range may be determined appropriately based on the average speeds or the like of the vehicles 10. Based on positional information for the first user and positional information for the plurality of vehicles 10, the controller 33 detects a vehicle 10 that is located within the setting range from the position of the first user.

When a plurality of vehicles 10 located within the aforementioned setting range has been detected, the controller 33 may determine a vehicle 10 that is located closest to the position of the first user, as the vehicle 10 to which the signal that gives instructions to help the first user is to be transmitted.

Upon determining the vehicle 10 to which the signal that gives instructions to help the first user is to be transmitted, the controller 33 transmits the signal that gives instructions to help the first user to the vehicle 10 via the network 2 using the communication interface 31. The controller 33 may transmit, to the vehicle 10 via the network 2, positional information for the first user and medical condition information for the first user, together with the signal that gives instructions to help the first user. With the above configuration, the controller 17 of the vehicle 10 can receive the positional information for the first user and the medical condition information for the first user, together with the signal that gives instructions to help the first user, from the information processing apparatus 30 via the network 2 using the communication interface 12. Further, the controller 17 can cause the output interface 15 to output the positional information for the first user and the medical condition information for the first user. As a result of the positional information for the first user being outputted from the output interface 15, a doctor can drive the vehicle 10 to the first user. Further, as a result of the medical condition information for the first user being outputted from the output interface 15, the doctor can speculate on the cause or the like of an abnormal physical condition of the first user based on the medical condition information for the first user.

The controller 33 may transmit, to the determined vehicle 10 to which the signal that gives instructions to help the first user is to be transmitted, information on how to use a medication that has been determined to be used for the first user as described above, via the network 2 using the communication interface 31. With the above configuration, the controller 17 of the vehicle 10 can receive the information on how to use the medication from the information processing apparatus 30 via the network 2 using the communication interface 12. Further, the controller 17 can cause the output interface 15 to output the information on how to use the medication. As a result of the information on how to use the medication being outputted from the output interface 15, the doctor can understand how to use the medication.

The controller 33 may execute the processing of Example 2 in combination with the processing of Example 1.

### EXAMPLE 3: VEHICLE DETERMINATION PROCESSING

When a plurality of vehicles 10 located within the aforementioned setting range has been detected, the controller 33 acquires specialty information for a plurality of doctors. The controller 33 identifies at least one doctor among the plurality of doctors, based on the specialty information for the plurality of doctors and medical condition information for the first user.

For example, it is assumed that the medical condition information for the first user is information indicating the name of a disease and that the specialty information for the doctors indicates which specialties the doctors hold. In this case, information on specialists who generally see diseases with names that are indicated by medical condition information for users may be stored in the memory 32. The controller 33 retrieves, from the memory 32, information on a specialist who generally sees a disease with the name that is indicated by the medical condition information for the first user. The controller 33 identifies a doctor who holds the same specialty information as the retrieved specialist information from the plurality of doctors.

The controller 33 determines whether a vehicle 10 that the identified doctor is on board exists among the plurality of vehicles 10 located within the aforementioned setting range.

If it is determined that a vehicle 10 that the identified doctor is on board exists among the plurality of vehicles 10 located within the aforementioned setting range, the controller 33 determines the vehicle 10 that the identified doctor is on board as a vehicle 10 to which a signal that gives instructions to help the first user is to be transmitted. On the other hand, if it is determined that a vehicle 10 that the identified doctor is on board does not exist among the plurality of vehicles 10 located within the aforementioned setting range, the controller 33 determines the vehicle 10 to which the signal that gives instructions to help the first user is to be transmitted in a manner similar to the above Example 2. That is, the controller 33 determines a vehicle 10 in the plurality of vehicles 10 within the aforementioned setting range that is closest to the position of the first user, as the vehicle 10 to which the signal that gives instructions to help the first user is to be transmitted.

For example, it is assumed that the first user is the user U2. In the configuration illustrated in FIG. 4, it is assumed that the controller 33 has detected the vehicle 10A with the positional information “AAA” and the vehicle 10C with the positional information “CCC”, as vehicles 10 located within a setting range from the position of the user U2. Further, the controller retrieves, from the memory 32, the information indicating “otolaryngologist” as information indicating a specialist who generally sees the disease “bronchial asthma” indicated by medical condition information for the user U2. In this case, the controller 33 determines a vehicle 10C that the doctor D3 with the specialty information “otolaryngologist” is on board as the vehicle 10 to which the signal that gives instructions to help the first user is to be transmitted.

Upon determining the vehicle 10 to which the signal that gives instructions to help the first user is to be transmitted, the controller 33 transmits, to the vehicle 10, the signal that gives instructions to help the first user via the network 2 using the communication interface 31, in a manner similar to the above Example 2. As is the case with the above Example 2, the controller 33 may transmit positional information for the first user and medical condition information for the first user, together with the signal that gives instructions to help the first user, to the vehicle 10 via the network 2. As is the case with the above Example 2, the controller 33 may transmit, to the determined vehicle 10 to which the signal that gives instructions to help the first user is to be transmitted, information on how to use a medication that has been determined to be used for the first user as described above, via the network 2 using the communication interface 31.

The controller 33 may execute the processing of Example 3 in combination with the processing of Example 1.

### EXAMPLE 4: MEDICATION AND VEHICLE DETERMINATION PROCESSING

When a plurality of vehicles 10 located within the aforementioned setting range has been detected, the controller 33 selects a medication from a plurality of medications, based on medical condition information for the first user and efficacy information for the plurality of medications, in a manner similar to the above Example 1. The controller 33 determines whether a vehicle 10 that a doctor who is presumed to be in possession of the selected medication is on board exists among the plurality of vehicles 10 located within the aforementioned setting range. In a case in which identification information for the selected medication is associated with doctor identification information, the controller 33 infers that the doctor is in possession of the medication.

If it is determined that a vehicle 10 that a doctor who is presumed to be in possession of the selected medication is on board exists among the plurality of vehicles 10 located within the aforementioned setting range, the controller 33 determines the vehicle 10 that the doctor who is presumed to be in possession of the selected medication is on board as a vehicle to which a signal that gives instructions to help the first user is to be transmitted. The controller 33 also determines that the selected medication is to be used for the first user. The controller 33 transmits a signal that gives instructions to provide the medication and the signal that gives instructions to help the first user, to the determined vehicle 10 to which the signal that gives instructions to help the first user is to be transmitted via the network 2 using the communication interface 31. The controller 33 may transmit positional information for the first user, the medical condition information for the first user, and information on how to use the determined medication to be used for first user, together with the above signals, to the vehicle 10 via the network 2 using the communication interface 31.

On the other hand, if it is determined that a vehicle 10 that a doctor who is presumed to be in possession of the selected medication is on board does not exist among the plurality of vehicles 10 located within the above setting range, the controller 33 determines a vehicle 10 located closest to the position of the first user as the vehicle to which the signal that gives instructions to help the first user is to be transmitted, in a manner similar to the above Example 2. Further, as is the case with the above Example 1, the controller 33 determines a medication in the plurality of medications that is selected based on the medical condition information for the first user and the efficacy information for the plurality of medications and that is located closest to the position of the first user, as a medication to be used for the first user. Upon determining the vehicle 10 to which the signal that gives instructions to help the first user is to be transmitted, the controller 33 transmits the signal that gives instructions to help the first user to the vehicle 10 via the network 2 using the communication interface 31, in a manner similar to the above Example 2. As is the case with the above Example 2, the controller 33 may transmit positional information for the first user and medical condition information for the first user, together with the signal that gives instructions to help the first user, to the vehicle 10 via the network 2. Further, upon determining the medication to be used for the first user, the controller 33 transmits a signal that gives instructions to provide the medication and positional information for the first user to a vehicle 10 or to a terminal apparatus 20 using the communication interface 21, in a manner similar to the above Example 1.

For example, it is assumed that the first user is the user U2. It is assumed that the controller 33 has detected the vehicle 10A with the positional information “AAA” and the vehicle 10C with the positional information “CCC”, as vehicles 10 located within a setting range from the position of the user U2. The controller 33 selects the medication with the identification information “XXX” from the plurality of medications. The controller 33 infers that the doctor D1 is in possession of the medication with the identification information “XXX.” The controller 33 determines the vehicle 10A as the vehicle to which the signal that gives instructions to help the first user is to be transmitted.

### Additional Example of Determination Processing

Upon detection of an abnormal physical condition of the first user, the controller 33 may determine which one of a plurality of medical devices as illustrated in FIG. 5 is to be used for the first user. The controller 33 determines which one of the plurality of medical devices is to be used for the first user, based on positional information for the first user, medical condition information for the first user, positional information for the plurality of medical devices, information on the types of the plurality of medical devices, and information on the dates and times when the plurality of medical devices can be used. An example of this processing is described below.

The controller 33 obtains a detection time at which an abnormal physical condition of the first user has been detected, by the timekeeping function of the controller 33. For example, in a case in which the controller 33 detects an abnormal physical condition of the first user by receiving a signal indicating the abnormal physical condition of the user from the terminal apparatus 20, the controller 33 obtains a time at which the signal indicating the abnormal physical condition of the user has been received as the detection time. The controller 33 identifies at least one medical device that can be used from the detection time to a predetermined setting time from a plurality of medical devices, based on information on the dates and times when the medical devices can be used. The setting time may be determined appropriately by estimating the amount of time required to help the user.

Upon identifying the at least one medical device that can be used, the controller 33 selects a medical device from the at least one medical device that can be used, based on medical condition information for the first user and information on the types of the medical devices. For example, it is assumed that the user medical condition information is information indicating the name of a disease. In this case, information indicating the names of diseases and information indicating the types of medical devices used to treat the diseases with these names may be stored in the memory 32 in association with each other. The controller 33 retrieves, from the memory 32, information indicating the type of a medical device that is associated with information indicating the name of a disease corresponding to the medical condition information for the first user, and selects a medical device that is of the same type as the retrieved type of medical device.

For example, it is assumed that the first user is the user U2. It is assumed that information indicating the name of the disease “bronchial asthma” and information indicating the type of the medical device “ventilator” are stored in the memory 32 in association with each other. In this case, the controller 33 retrieves information indicating the type of the medical device “ventilator” from the memory 32. The controller 33 selects the device M2 that is of the same type as the retrieved type “ventilator.”

The controller 33 determines, with respect to the selected medical device, a medical device that is located closest to the position of the first user as a device to be used for the first user. The controller 33 may identify the medical device that is located closest to the position of the first user, based on positional information for the medical devices and positional information for the first user.

The controller 33 transmits positional information for the determined medical device to be used for the first user, together with the positional information for the first user, to a terminal apparatus 20 or a vehicle 10 that is closest to the position of the determined medical device, using the communication interface 31.

With the above configuration, if the positional information or the like for the medical device is transmitted to a terminal apparatus 20, the controller 26 of the terminal apparatus 20 can receive the positional information for the medical device and the positional information for the first user from the information processing apparatus 30 via the network 2 using the communication interface 21. Further, the controller 26 can cause the output interface 24 to output the positional information for the medical device and the positional information for the first user. As a result of the positional information for the medical device and the positional information for the first user being outputted from the output interface 24, a user can understand the position of the medical device and the position of the first user. After travelling to the medical device, the user can travel to the first user with the medical device.

With the above configuration, if the positional information or the like for the medical device is transmitted to a vehicle 10, the controller 17 of the vehicle 10 can receive the positional information for the medical device and the positional information for the first user from the information processing apparatus 30 via the network 2 using the communication interface 12. Further, the controller 17 can cause the output interface 15 to output the positional information for the medical device and the positional information for the first user. As a result of the positional information for the medical device and the positional information for the first user being outputted from the output interface 15, a doctor can understand the position of the medical device and the position of the first user. After travelling to the medical device, the doctor can travel to the first user with the medical device.

The controller 33 may transmit information on how to use the determined medical device to be used for the first user to a vehicle 10 to which a signal that gives instructions to help the first user has been determined to be transmitted as described above, via the network 2 using the communication interface 31. With the above configuration, the controller 17 of the vehicle 10 can receive the information on how to use the medical device from the information processing apparatus 30 via the network 2 using the communication interface 12. Further, the controller 17 can cause the output interface 15 to output the information on how to use the medical device. As a result of the information on how to use the medical device being outputted from the output interface 15, a doctor can understand how to use the medical device that a user or another doctor has brought to the first user.

### Additional Processing

The controller 33 may communicably connect a vehicle 10 to which a signal that gives instructions to help the first user has been transmitted and a terminal apparatus used by a healthcare professional. For example, the healthcare professional is in a distant place from the first user. The healthcare professional is a person who is engaged in health care. The healthcare professional is not limited to a doctor. Information on a connection destination of the terminal apparatus used by the healthcare professional may be stored in the memory 32.

The controller 33 identifies at least one healthcare professional among a plurality of healthcare professionals, based on information for the plurality of healthcare professionals and medical condition information for the first user. The controller 33 communicably connects a terminal apparatus used by the identified healthcare professional and the vehicle 10 to which the signal that gives instructions to help the first user has been transmitted. For example, it is assumed that the medical condition information for the first user is information indicating the name of a disease. In this case, information indicating the names of diseases and information indicating healthcare professionals who specialize in diseases with these names may be stored in the memory 32 in association with each other. The controller 33 identifies the at least one healthcare professional among the plurality of healthcare professionals, by retrieving, from the memory 32, information on at least one healthcare professional who specializes in a disease corresponding to the name of a disease indicated by the medical condition information for the first user.

The controller 33 may identify at least one healthcare professional among the plurality of healthcare professionals, based on information on the plurality of healthcare professionals and diagnosis information for the first user. The controller 33 receives the diagnosis information for the first user from the vehicle 10 to which the signal that gives instructions to help the first user has been transmitted, via the network 2 using the communication interface 31. The diagnosis information for the first user is information indicating a symptom of the first user. The diagnosis information for the first user is transmitted from the vehicle 10 to the information processing apparatus 30 by a doctor who has traveled to the first user. For example, after seeing the first user, for instance, the doctor inputs diagnosis information for the first user through the input interface 14. Upon receiving the diagnosis information for the first user through the input interface 14, the controller 17 of the vehicle 10 transmits the diagnosis information for the first user to the information processing apparatus 30 via the network 2 using the communication interface 12. Here, information on symptoms and information on healthcare professionals who specialize in the symptoms may be stored in the memory 32 in association with each other. The controller 33 identifies the at least one healthcare professional among the plurality of healthcare professionals, by retrieving, from the memory 32, information on at least one healthcare professional who specializes in a symptom indicated by the diagnosis information for the first user.

When a plurality of healthcare professionals has been identified, the controller 33 may select, from the identified plurality of healthcare professionals, a healthcare professional who is close to the doctor on the vehicle 10 to which the signal that gives instructions to help the first user has been transmitted. The controller 33 may communicably connect a terminal apparatus used by the selected healthcare professional and the vehicle 10 to which the signal that gives instructions to help the first user has been transmitted. Identification information for doctors and information on healthcare professionals who are close to the doctors may be stored in the memory 32 in association with each other. The controller 33 selects the healthcare professional by retrieving, from the memory 32, information indicating a healthcare professional associated with identification information for the doctor on the vehicle 10 to which the signal that gives instructions to help the first user has been transmitted.

With a vehicle 10 and a terminal apparatus of a healthcare professional being communicably connected in this manner, a doctor can converse with the healthcare professional via the input interface 14 and the output interface 15. The doctor can converse with the healthcare professional and receive advice from the healthcare professional.

### Additional Processing

The controller may communicably connect a vehicle 10 to which a signal that gives instructions to help the first user is to be transmitted and a terminal apparatus used by a family member of the first user. Identification information for users and information on connection destinations of terminal apparatuses used by family members of the users may be stored in the memory 32 in association with each other. With the above configuration, a doctor can converse with a family member of the first user via the input interface 14 and the output interface 15 of the vehicle 10.

### Operations of Information Processing System

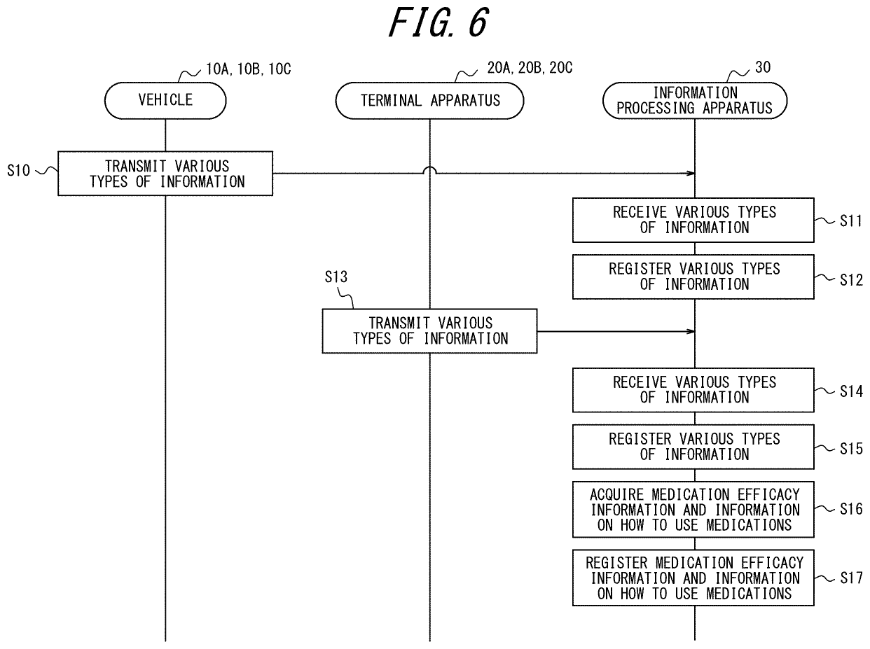

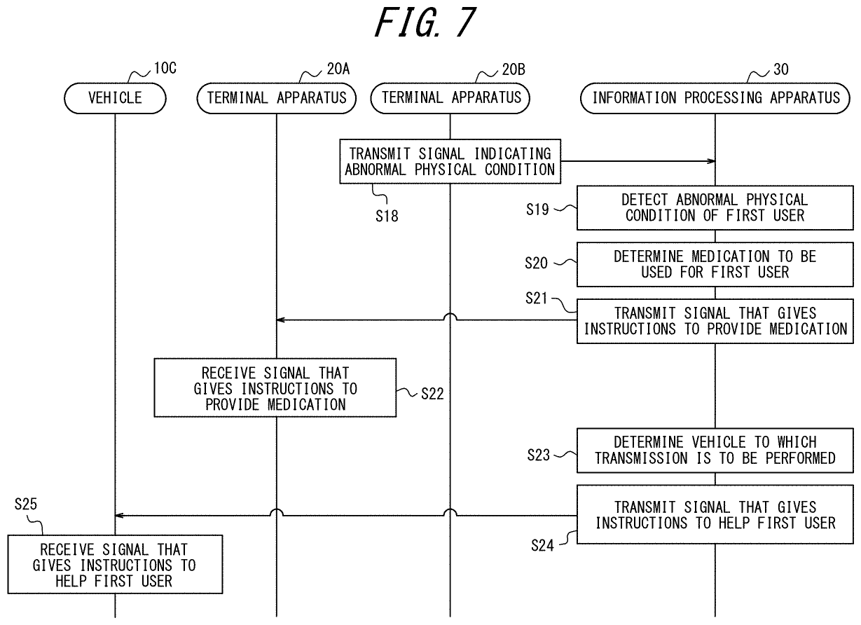

FIG. 6 and FIG. 7 are sequence diagrams illustrating examples of the operations of the information processing system 1 of FIG. 1. The operations may correspond to an example of an information processing method according to the present embodiment.

In each of the vehicles 10A to 10C, the controller 17 executes transmission processing for transmitting, to the information processing apparatus 30, various types of information to be registered on the information processing apparatus 30 (Step S10). In the information processing apparatus 30, the controller 33 receives, from each of the vehicles 10A to 10C, various types of information via the network 2 using the communication interface 31 (Step S11). In the information processing apparatus 30, the controller 33 executes registration processing for registering various types of information by storing the received various types of information in the memory 32 (Step S12).

In each of the terminal apparatuses 20A to 20C, the controller 26 executes transmission processing for transmitting, to the information processing apparatus 30, various types of information to be registered on the information processing apparatus 30 (Step S13). In the information processing apparatus 30, the controller 33 receives, from each of the terminal apparatuses 20A to 20C, various types of information via the network 2 using the communication interface 31 (Step S14). In the information processing apparatus 30, the controller 33 executes registration processing for registering various types of information by storing the received various types of information in the memory 32 (Step S15).

In the information processing apparatus 30, upon registration of medication identification information, the controller 33 acquires, for identification information for each medication registered, medication efficacy information and information on how to use the medication (Step S16). In the information processing apparatus 30, the controller 33 stores in the memory 32 the acquired medication efficacy information and information on how to use medications, thereby registering the medication efficacy information and the information on how to use the medications in association with the medical identification information (Step S17).

After executing the processing of step S17, the information processing system 1 proceeds to the processing of Step S18 as illustrated in FIG. 7.

In the terminal apparatus 20B, the controller 26 detects an abnormal physical condition of the user U2, and transmits a signal indicating the abnormal physical condition to the information processing apparatus 30 via the network 2 using the communication interface 31 (Step S18).

In the information processing apparatus 30, the controller 33 detects the abnormal physical condition of the user U2 by receiving the signal indicating the abnormal physical condition of the user from the terminal apparatus 20B via the network 2 using the communication interface 31 (Step S19).

In the information processing apparatus 30, the controller 33 determines which one of a plurality of medications is to be used for the user U2 as the first user (Step S20). In Step S20, the controller 33 determines that a medication with the identification “XXX” in possession of the user U1 is to be used for the user U2 as the first user. The controller 33 transmits, to the terminal apparatus 20A used by the user U1, a signal that gives instructions to provide the medication and positional information for the first user, via the network 2 using the communication interface 31 (Step S21).

In the terminal apparatus 20A, the controller 26 receives the signal that gives instructions to provide the medication and the positional information for the first user, from the information processing apparatus 30 via the network 2 using the communication interface 21 (Step S22). In the terminal apparatus 20A, the controller 26 causes the output interface 24 to output information that gives instructions to provide the medication and the positional information for the first user.

In the information processing apparatus 30, the controller 33 determines to which vehicle 10, among a plurality of vehicles 10, a signal that gives instructions to help that first user is to be transmitted (Step S23). In the processing of Step S23, the controller 33 determines the vehicle 10C among the vehicles 10A to 10C as the vehicle 10 to which the signal that gives instructions to help the first user is to be transmitted. The controller 33 transmits positional information for the first user, together with the signal that gives instructions to help the first user, to the vehicle 10C via the network 2 using the communication interface 31 (Step S24).

In the vehicle 10C, the controller 17 receives the positional information for the first user, together with the signal that gives instructions to help the first user, from the information processing apparatus 30 via the network 2 using the communication interface 12 (Step S25). Upon receiving the signal, the controller 17 of the vehicle 10C causes the output interface 15 to output information that gives instructions to help the first user and identification information for the first user.

Thus, upon detection of an abnormal physical condition of the first user, the controller 33 of the information processing apparatus 30 determines to which vehicle 10, among a plurality of vehicles 10, a signal that gives instructions to help the first user is to be transmitted, and also determines which one of a plurality of medications is to be used for the first user. Because a medication is used to help the first user, an abnormal physical condition of the first user can be reversed rapidly. Further, because medications in possession of users are registered on the information processing apparatus 30, the users can share medications in their possession upon detection of an abnormal physical condition of a user. According to the present embodiment, an improved information processing apparatus 30 is therefore provided.

While an embodiment of the present disclosure has been described with reference to the drawings and examples, it should be noted that various modifications and revisions may be implemented by those skilled in the art based on the present disclosure. Accordingly, such modifications and revisions are included within the scope of the present disclosure. For example, functions or the like included in each means, each step, or the like can be rearranged without logical inconsistency, and a plurality of means, steps, or the like can be combined into one or divided.

For example, in the above embodiment, it is assumed that various types of information are transmitted from a terminal apparatus 20 to the information processing apparatus 30 via the network 2. Various types of information may, however, be transmitted to the information processing apparatus 30 via the network 2 from a vehicle that a user is on board. For example, the vehicle that the user is on board transmits user identification information, user medical condition information, and medication identification information to the information processing apparatus 30 via the network 2. The above information transmitted from the vehicle to the information processing apparatus 30 may be stored in advance in the vehicle. Further, the vehicle that the user is on board acquires vehicle positional information as user positional information, and transmits the acquired user positional information, together with the user identification information, to the information processing apparatus 30 via the network 2. When the physical condition of the user has deteriorated due to a seizure or the like when the user is driving the vehicle, the user enters an input indicating an abnormal physical condition through an input interface of the vehicle. Upon receiving the input, the vehicle transmits a signal indicating the abnormal physical condition, together with the user identification information, to the information processing apparatus 30 via the network 2.

1. An information processing apparatus, comprising a communication interface and a controller, wherein
the controller is configured to:
based on positional information for a first user whose abnormal physical condition has been detected and positional information for a plurality of vehicles that a respective plurality of doctors are on board, determine to which one of the plurality of vehicles a signal that gives instructions to help the first user is to be transmitted; and
based on the positional information for the first user, medical condition information for the first user, positional information for a plurality of medications, and efficacy information for the plurality of medications, determine which one of the plurality of medications is to be used for the first user.

2. The information processing apparatus according to claim 1, wherein
as positional information for at least one medication included in the positional information for the plurality of medications, the controller is configured to use positional information for a second user who is presumed to be in possession of the at least one medication.

3. The information processing apparatus according to claim 2, wherein
the controller is configured to identify the second user who is presumed to be in possession of the at least one medication, based on user medical condition information.

4. The information processing apparatus according to claim 2, wherein
as positional information for at least one medication included in the positional information for the plurality of medications, the controller is configured to use positional information for a vehicle that a doctor who is presumed to be in possession of the at least one medication is on board.

5. The information processing apparatus according to claim 2, wherein
in a case in which positional information for the determined medication to be used for the first user is the positional information for the second user, the controller transmits a signal that gives instructions to provide the medication and the positional information for the first user to a terminal apparatus used by the second user using the communication interface, and
in a case in which the positional information for the determined medication to be used for the first user is positional information for a vehicle, the controller transmits a signal that gives instructions to provide the medication and the positional information for the first user to the vehicle using the communication interface.

6. The information processing apparatus according to claim 1, wherein
the controller is configured to determine a medication in the plurality of medications that is selected based on the medical condition information for the first user and the efficiency information for the plurality of medications and that is located closest to a position of the first user, as the medication to be used for the first user.

7. The information processing apparatus according to claim 1, wherein
the controller is configured to determine a vehicle in the plurality of vehicles that is located within a predetermined setting range from a position of the first user, as the vehicle to which transmission is to be performed.

8. The information processing apparatus according to claim 7, wherein
when a plurality of vehicles located within the setting range has been detected, the controller determines a vehicle located closest to the position of the first user as the vehicle to which transmission is to be performed.

9. The information processing apparatus according to claim 7, wherein
when a plurality of vehicles located within the setting range has been detected, the controller acquires specialty information for the plurality of doctors, and identifies at least one doctor among the plurality of doctors based on the specialty information for the plurality of doctors and the medical condition information for the first user, and
if it is determined that a vehicle that the determined doctor is on board exists among the plurality of vehicles located within the setting range, the controller determines the vehicle that the determined doctor is on board as the vehicle to which transmission is to be performed.

10. The information processing apparatus according to claim 7, wherein
when a plurality of vehicles located within the setting range has been detected, the controller selects a medication from the plurality of medications based on the medical condition information for the first user and the efficacy information for the plurality of medications, and
if it is determined that a vehicle that a doctor who is presumed to be in possession of the selected medication is on board exists among the plurality of vehicles located within the setting range, the controller determines the vehicle that the doctor who is presumed to be in possession of the selected medication is on board as the vehicle to which transmission is to be performed, and determines the selected medication as the medication to be used for the first user.

11. The information processing apparatus according to claim 1, wherein
the controller is configured to transmit, to the vehicle to which transmission is to be performed, the positional information for the first user and the medical condition information for the first user, together with the signal that gives instructions to help the first user, using the communication interface.

12. The information processing apparatus according to claim 1, wherein
the controller is configured to transmit, to the vehicle to which transmission is to be performed, information on how to use the determined medication to be used for the first user using the communication interface.

13. The information processing apparatus according to claim 1, wherein
the controller is configured to:
based on information on a plurality of healthcare professionals and on the medical condition information for the first user or diagnosis information for the first user received using the communication interface from the vehicle to which transmission has been performed, identify at least one healthcare professional among the plurality of healthcare professionals; and
communicably connect the vehicle to which transmission has been performed and a terminal apparatus used by the identified healthcare professional.

14. The information processing apparatus according to claim 1, wherein
the controller is configured to determine which one of a plurality of medical devices is to be used for the first user, based on the positional information for the first user, the medical condition information for the first user, positional information for the plurality of medical devices, information on types of the plurality of medical devices, and information on dates and times when the plurality of medical devices can be used.

15. The information processing apparatus according to claim 14, wherein
the controller is configured to transmit positional information for the determined medical device to be used for the first user and the positional information for the first user to a terminal apparatus or a vehicle that is closest to a position of the determined medical device using the communication interface.

16. The information processing apparatus according to claim 1, wherein
the controller is configured to detect the abnormal physical condition of the first user by receiving a signal indicating the abnormal physical condition from a terminal apparatus using the communication interface.

17. The information processing apparatus according to claim 1, wherein
the controller is configured to receive positional information and medical condition information for a plurality of users including the first user from a plurality of terminal apparatuses using the communication interface.

18. The information processing apparatus according to claim 1, wherein
the controller is configured to receive the positional information for the plurality of vehicles from the respective plurality of vehicles using the communication interface.

19. A non-transitory computer readable medium storing a program configured to cause an information processing apparatus to execute operations comprising:
based on positional information for a first user whose abnormal physical condition has been detected and positional information for a plurality of vehicles that a respective plurality of doctors are on board, determining to which one of the plurality of vehicles a signal that gives instructions to help the first user is to be transmitted; and
based on the positional information for the first user, medical condition information for the first user, positional information for a plurality of medications, and efficacy information for the plurality of medications, determining which one of the plurality of medications is to be used for the first user.

20. An information processing method comprising:
based on positional information for a first user whose abnormal physical condition has been detected and positional information for a plurality of vehicles that a respective plurality of doctors are on board, determining to which one of the plurality of vehicles a signal that gives instructions to help the first user is to be transmitted; and
based on the positional information for the first user, medical condition information for the first user, positional information for a plurality of medications, and efficacy information for the plurality of medications, determining which one of the plurality of medications is to be used for the first user.
